(19) United States
(12) Patent Application Publication
(10) Pub. No.: US 2018/0161476 A1
(43) Pub. Date: Jun. 14, 2018

(54) HYDROGEL FOAM PATCH FOR OXYGEN DELIVERY AND METHOD OF MANUFACTURE

(71) Applicant: Kimberly-Clark Worldwide, Inc., Neenah, WI (US)

(72) Inventors: John Gavin MacDonald, Decatur, GA (US); Brian J. Cuevas, Cumming, GA (US); Dave Soerens, Neenah, WI (US)

(21) Appl. No.: 15/571,064

(22) PCT Filed: Jun. 30, 2015
(86) PCT No.: PCT/US2015/038425

§ 371 Date: Nov. 1, 2017

(51) Int. Cl. A61L 15/44; A61L 15/60; A61L 15/42; A61L 15/18; A61L 15/24; C08J 9/28

(52) U.S. Cl. CPC A61L 15/44; A61L 15/60; A61L 15/425; A61L 15/18; A61L 15/24; C08J 2201/0504; C08J 2205/022; A61L 2300/10; C08J 2205/052; C08J 2333/02; C08J 9/28

(57) ABSTRACT

The present disclosure is directed to a closed cell foam matrix for delivering oxygen containing a superabsorbent material oxygen entrapped within the superabsorbent material. The superabsorbent material has at least 15 percent by mass monoethylenically unsaturated carboxylic, sulphonic or phosphoric acid or salts thereof, an acrylate or methacrylate ester that contains an alkoxysilane functionality, and a copolymerizable hydrophilic glycol containing ester monomer. To produce the closed cell foam matrix for delivering oxygen, an alkali hydroxide catalyst is added to the superabsorbent material to form a hydrogel layer. Then, an oxygen precursor is added to the hydrogel layer. The hydrogel layer is heated to produce oxygen by reacting the alkali hydroxide catalyst and the oxygen precursor thereby entrapping the oxygen in the formed closed cell foam matrix.

### BACKGROUND

In the United States, non-healing wounds affect around 3-6 million patients, accounting for more than 25 billion dollars spent on treatment each year. Although non-healing wounds are frequently reported in diabetic patients, intrinsic aging is another risk factor that delays the healing process. Cellular senescence, chronic inflammation and alteration of skin homeostasis may partially explain the impaired responses in the elderly. Considering that the process of wound healing requires a high energy level to support rapid cell growth and metabolism; oxygen plays a crucial role in acceleration of wound closure and may be applicable for promotion of elderly skin health.

Damage or destruction of the blood supply to a region of living tissue quickly leads to compromised tissue. One of the critical functions of an adequate blood supply is the provision of dissolved gases to the site, such as oxygen. For example, wounds to bodily tissues are accompanied by damage or destruction of the natural blood supply that transports oxygen and nutrients that are necessary to support the healing process. Oxygen has been shown to have therapeutic effect in healing of wounds and in preventing growth of anaerobic bacteria etc. While oxygen may be available from air for direct dissolution into wound fluids, availability of topically dissolved oxygen is preferred can accelerate the benefits of healing.

Thus, there is a need for a closed cell oxygen releasing foam that is practical to manufacture and handle. There is also a need for a practical and economical method of manufacturing such a closed cell oxygen releasing foam. Methods and compositions are needed that can provide oxygen to a wound.

### SUMMARY

The present disclosure is directed to a closed cell foam matrix for delivering oxygen containing a superabsorbent material oxygen entrapped within the superabsorbent material. The superabsorbent material has at least 15 percent by mass monoethylenically unsaturated carboxylic, sulphonic or phosphoric acid or salts thereof, an acrylate or methacrylate ester that contains an alkoxysilane functionality, and a copolymerizable hydrophilic glycol containing ester monomer. Desirably, an aqueous solution of an oligomeric polyacrylic acid having a silanol cross-linker covalently bonded to the backbone chain of a polyacrylic acid is used for the superabsorbent material described herein.

The closed cell foam could be formed in a variety of shapes and forms; such as, in a sheet or layer; coating infused on to a nonwoven matrix; extruded fibers; coating on fibers, powder. All of these forms would be capable of releasing oxygen.

To produce the closed cell foam matrix for delivering oxygen, an alkali hydroxide catalyst is added to the superabsorbent material to form a hydrogel layer. Examples of alkali hydroxide catalyst that can be used include, but are not limited to, sodium hydroxide, lithium hydroxide, potassium hydroxide, magnesium hydroxide, calcium hydroxide, and combinations thereof. In desirable embodiments, the alkali hydroxide catalyst comprises sodium hydroxide. Suitably, the amount of the alkali hydroxide catalyst that is added may be between about 0.5 percent to about 3 percent by weight relative to the weight of the liquid superabsorbent polymer composition.

After formation of the gel, an oxygen precursor is added to the hydrogel layer. Examples of an oxygen precursor that can be used include, but are not limited to, hydrogen peroxide, ammonium peroxide, sodium peroxide, urea peroxide complex, potassium percarbonate and combinations thereof. In desirable embodiments, the oxygen precursor comprises hydrogen peroxide. Suitably, the amount of the oxygen precursor that is added may be between about 15 percent to about 25 percent by weight relative to the weight of the liquid superabsorbent polymer composition.

After the oxygen precursor is added, the hydrogel layer is heated to produce oxygen by reacting the alkali hydroxide catalyst and the oxygen precursor thereby entrapping the oxygen in the formed closed cell foam matrix.

The present disclosure will be more fully understood, and further features will become apparent, when reference is made to the following detailed description and the accompanying drawings. The drawings are merely representative and are not intended to limit the scope of the claims.

### DETAILED DESCRIPTION

Compositions, methods and devices for the delivery of gases, preferably oxygen, or other active agents, to a localized environment are disclosed herein. Preferably, devices comprise matrices that can deliver known amounts of oxygen. The desirable embodiments are used in methods of treatment of compromised tissues and for methods of preserving life and maintaining the state of extracted tissues or organs. Compromised tissue as used herein can be one or more tissues and includes any organism, organ system, organ, tissue, cells or cellular components that is not in its normal metabolic state. For example, it means any tissue that has an abnormal blood supply, such as that caused by ischemic conditions, hypoxic conditions, infarction, occlusions, blockages, or trauma. It also includes wounds and damage to structural components. Also in the elderly skin tears, bed sores and bruises.

The present disclosure is directed to a closed cell foam matrix for delivering oxygen containing a superabsorbent material oxygen entrapped within the superabsorbent material. The superabsorbent material has at least 15 percent by mass monoethylenically unsaturated carboxylic, sulphonic or phosphoric acid or salts thereof, an acrylate or methacrylate ester that contains an alkoxysilane functionality, and a copolymerizable hydrophilic glycol containing ester monomer.

To produce the closed cell foam matrix for delivering oxygen, an alkali hydroxide catalyst is added to the superabsorbent material to form a hydrogel layer. Then, an oxygen precursor is added to the hydrogel layer. The hydrogel layer is heated to produce oxygen by reacting the alkali hydroxide catalyst and the oxygen precursor thereby entrapping the oxygen in the formed closed cell foam matrix. It can also be used where the alkali hydroxide catalyst and the oxygen precursor are both added to the superabsorbent material and then poured into forms and heated to make foamed samples, or infused or coated on a nonwoven and heated, or extruded into fibers which are then heat treated to make foamed fibers.

The compositions, methods and devices are used for the treatment of compromised tissues. A desirable embodiment comprises compositions and methods for treating compromised tissue comprising tissue contact materials that entrap oxygen within closed cell foam-like material capable of providing or maintaining optimal oxygen tension at a compromised tissue site while absorbing excess fluid and optimizing the microenvironment to facilitate tissue repair and regeneration if needed. In addition, desirable devices have superior wound exudate/moisture absorption capabilities. In certain embodiments, the methods, compositions and devices further comprise active agents incorporated therein for release at the site. In a further desirable embodiment, the closed cell foam-like material matrix composition comprises a flexible absorbent binder distributed evenly throughout the network. The matrices of this desirable embodiment provide a reliable and efficient means for maintaining oxygen tension, delivering active agents to the wound while at the same time providing a superior moisture regulation capacity.

The tissue contact material devices are not restricted by form or shape. The devices may be constructed in sheet style formats of various dimensions. Similarly, the materials can be molded to conform to various shapes and contours as required by the intended use. The present disclosure is directed to compositions, methods and devices for the delivery of active agents, including oxygen. Desirable embodiments are directed to delivery of oxygen to compromised tissue. An example of desirable embodiments for treatment of compromised tissues is the treatment of wounds. This example is for illustration, and should not be used in a limiting sense, and such desirable embodiments can be used for treatment of other types of compromised tissue.

As discussed above, the closed cell foam is produced with a superabsorbent polymer material. A superabsorbent polymer material suitable for use herein is described as a superabsorbent binder polymer solution in U.S. Pat. Nos. 6,849,685 to Soerens et al., 7,312,286 to Lang et al., and U.S. Pat. No. 7,335,713 to Lang et al., the entirety of each of these references is herein incorporated by reference. The superabsorbent binder polymer solution described therein is capable of post-application, moisture-induced crosslinking. Whereas most superabsorbent polymers require the addition of an internal crosslinker to reinforce the polymer, the superabsorbent polymer material used herein does not require the addition of a crosslinking agent because the organic monomers act as an internal crosslinker. The internal crosslinker allows the superabsorbent polymer material to be formed by coating the water-soluble precursor polymer onto the substrate and then removing the water to activate the latent crosslinker.

An absorbent binder composition that may be used as a superabsorbent polymer material described herein. The absorbent binder composition disclosed in Soerens et al. is a monoethylenically unsaturated polymer and an acrylate or methacrylate ester that contains an alkoxysilane functionality that is particularly suitable for use in manufacturing absorbent articles. Also described in Soerens et al. is a method of making the absorbent binder composition that includes the steps of preparing a monomer solution, adding the monomer solution to an initiator system, and activating a polymerization initiator within the initiator system reported an alcohol-based, water-soluble binder composition. “Monomer(s)” as used herein includes monomers, oligomers, polymers, mixtures of monomers, oligomers and/or polymers, and any other reactive chemical species which are capable of co-polymerization with monoethylenically unsaturated carboxylic, sulphonic or phosphoric acid or salts thereof. Ethylenically unsaturated monomers containing a trialkoxysilane functional group are appropriate for this invention and are desired. Desired ethylenically unsaturated monomers include acrylates and methacrylates, such as acrylate or methacrylate esters that contain an alkoxysilane functionality.

The superabsorbent binder polymer composition disclosed in the references noted above is the reaction product of at least 15 percent by mass monoethylenically unsaturated carboxylic, sulphonic or phosphoric acid or salts thereof, an acrylate or methacrylate ester that contains an alkoxysilane functionality which, upon exposure to water, forms a silanol functional group which condenses to form a crosslinked polymer, a copolymerizable hydrophilic glycol containing ester monomer; and/or, a plasticizer.

The monoethylenically unsaturated monomer is desirably acrylic acid. Other suitable monomers include carboxyl group-containing monomers: for example monoethylenically unsaturated mono or poly-carboxylic acids, such as (meth)acrylic acid (meaning acrylic acid or methacrylic acid; similar notations are used hereinafter), maleic acid, fumaric acid, crotonic acid, sorbic acid, itaconic acid, and cinnamic acid; carboxylic acid anhydride group-containing monomers: for example monoethylenically unsaturated polycarboxylic acid anhydrides (such as maleic anhydride); carboxylic acid salt-containing monomers: for example water-soluble salts (alkali metal salts, ammonium salts, amine salts, and the like) of monoethylenically unsaturated mono- or poly-carboxylic acids (such as sodium (meth)acrylate, trimethylamine (meth)acrylate, triethanolamine (meth)acrylate), sodium maleate, methylamine maleate; sulfonic acid group-containing monomers: for example aliphatic or aromatic vinyl sulfonic acids (such as vinylsulfonic acid, allyl sulfonic acid, vinyltoluenesulfonic acid, styrene sulfonic acid), (meth)acrylic sulfonic acids [such as sulfopropyl (meth)acrylate, 2-hydroxy-3-(meth)acryloxy propyl sulfonic acid]; sulfonic acid salt group-containing monomers: for example alkali metal salts, ammonium salts, amine salts of sulfonic acid group containing monomers as mentioned above; and/or amide group-containing monomers: vinylformamide, (meth)acrylamide, N-alkyl (meth)acrylamides (such as N-methylacrylamide, N-hexylacrylamide), N,N-dialkyl (meth)acryl amides (such as N,N-dimethylacrylamide, N,N-di-n-propylacrylamide), N-hydroxyalkyl (meth)acrylamides [such as N-methylol (meth)acrylamide, N-hydroxyethyl (meth)acrylamide], N,N-dihydroxyalkyl (meth)acrylamides [such as N,N-dihydroxyethyl (meth)acrylamide], vinyl lactams (such as N-vinylpyrrolidone).

Suitably, the amount of monoethylenically unsaturated carboxylic, sulphonic or phosphoric acid or salts thereof relative to the weight of the superabsorbent binder polymer composition may range from about 15 percent to about 99.9 percent by weight. The acid groups are desirably neutralized to the extent of at least about 25 mol percent, that is, the acid groups are preferably present as sodium, potassium or ammonium salts. The degree of neutralization is preferably at least about 50 mol percent.

One of the issues in preparing water-soluble polymers is the amount of the residual monoethylenically unsaturated monomer content remaining in the polymer. For applications in personal hygiene it is required the amount of residual monoethylenically unsaturated monomer content of the superabsorbent polymer composition be less than about 1000 ppm, and more preferably less than 500 ppm, and even more preferably less than 100 ppm. U.S. Pat. No. 7,312,286 discloses at least one method by which an absorbent binder composition may be manufactured so that the residual monoethylenically unsaturated monomer content is at least less than 1000 parts per million. The analysis of residual monoethylenically unsaturated monomer is determined according to the Residual Monoethylenically Unsaturated Monomer Test which is disclosed in U.S. Pat. No. 7,312,286. More specifically, the residual monoethylenically unsaturated monomer analysis is carried out using solid film obtained from the polymer solution or superabsorbent composition. By way of example for this test description, the monoethylenically unsaturated monomer is acrylic acid. High performance liquid chromatography (HPLC) with a SPD-IOAvp Shimadzu UV detector (available from Shimadzu Scientific Instruments, having a place of business in Columbia, Md., U.S.A) is used to determine the residual acrylic acid monomer content. To determine the residual acrylic acid monomer, about 0. 5 grams of cured film is stirred in 100 ml of a 0. 9% NaCl-solution for 16 h using a 3. 5 cm L×0. 5 cm W magnetic stirrer bar at 500 rpm speed. The mixture is filtered and the filtrate is then passed through a Nucleosil C8 100A reverse phase column (available from Column Engineering Incorporated, a business having offices located in Ontario, Calif., U.S.A.) to separate the acrylic acid monomer. The acrylic acid monomer elutes at a certain time with detection limit at about 10 ppm. The peak area of resulting elutes calculated from the chromatogram is then used to calculate the amount of residual acrylic acid monomer in the film. Initially, a calibration curve was generated by plotting the response area of pure acrylic acid elutes against its known amount (ppm). A linear curve with a correlation coefficient of greater than 0. 996 was obtained.

Desirably, an aqueous solution of an oligomeric polyacrylic acid having a silanol cross-linker covalently bonded to the backbone chain of a polyacrylic acid is used for the superabsorbent material described herein.

To produce the closed cell foam matrix for delivering oxygen, an alkali hydroxide catalyst is added to the superabsorbent material to form a hydrogel layer. Examples of alkali hydroxide catalyst that can be used include, but are not limited to, sodium hydroxide, lithium hydroxide, potassium hydroxide, magnesium hydroxide, calcium hydroxide, and combinations thereof. In desirable embodiments, the alkali hydroxide catalyst comprises sodium hydroxide. Suitably, the amount of the alkali hydroxide catalyst that is added may be between about 0.5 percent to about 3 percent by weight relative to the weight of the liquid superabsorbent polymer composition.

After formation of the gel, an oxygen precursor is added to the hydrogel layer. Examples of oxygen precursor that can be used include, but are not limited to, hydrogen peroxide, ammonium peroxide, sodium peroxide, urea peroxide complex, potassium percarbonate and combinations thereof. In desirable embodiments, the oxygen precursor comprises hydrogen peroxide. Suitably, the amount of the oxygen precursor that is added may be between about 15 percent to about 25 percent by weight relative to the weight of the liquid superabsorbent polymer composition.

After the oxygen precursor is added, the hydrogel layer is heated to produce oxygen by reacting the alkali hydroxide catalyst and the oxygen precursor thereby entrapping the oxygen in a formed closed cell foam matrix. In preferred embodiments, the hydrogel layer is heated at a temperature of at least 50 degrees Celsius.

In another embodiment, a molar ratio of the alkali hydroxide catalyst to the oxygen precursor is in the range of 1.0:0.9 to 0.9:1.0 with the alkalki hydroxide catalyst having an additional amount to neutralize the acid component superabsorbent material.

Optionally, active agents are incorporated into the closed cell foam matrix. Active agents and their effects are known by those skilled in the art and methods for including these agents into the matrices are taught herein. The present invention contemplates the inclusion of one or more active agents, depending on the intended use. The compositions and devices may include one agent, such as oxygen, or may include multiple agents. For example, if the device is a matrix gel sheet placed in a tissue culture dish and is used to provide oxygen to the growing cells, the active agents include oxygen and any other agents that aid the cells, such as antimicrobials to maintain sterility, or growth factors to aid in cell growth.

If the devices are used for topical treatments, such as treatments for compromised tissues, the devices comprise active agents that aid in treatment of compromised tissues. For example, the devices are used for the treatment of wounds, in skin healing or for cosmetic applications. The active agents aid and improve the wound healing process, and may include gases, anti-microbial agents, including but not limited to, anti-fungal agents, anti-bacterial agents, anti-viral agents and anti-parasitic agents, mycoplasma treatments, growth factors, proteins, nucleic acids, angiogenic factors, anaesthetics, mucopolysaccharides, metals and other wound healing agents.

Active agents include, but are not limited to, gases, such as nitrogen, carbon dioxide, and noble gases, pharmaceuticals, chemotherapeutic agents, herbicides, growth inhibitors, anti-fungal agents, anti-bacterial agents, anti-viral agents and anti-parasitic agents, mycoplasma treatments, growth factors, proteins, nucleic acids, angiogenic factors, anaesthetics, mucopolysaccharides, metals, wound healing agents, growth promoters, indicators of change in the environment, enzymes, nutrients, vitamins, minerals, carbohydrates, fats, fatty acids, nucleosides, nucleotides, amino acids, sera, antibodies and fragments thereof, lectins, immune stimulants, immune suppressors, coagulation factors, neurochemicals, cellular receptors, antigens, adjuvants, radioactive materials, and other agents that effect cells or cellular processes.

Examples of anti-microbial agents that can be used include, but are not limited to, isoniazid, ethambutol, pyrazinamide, streptomycin, clofazimine, rifabutin, fluoroquinolones, ofloxacin, sparfloxacin, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, clindamycin, lincomycin, pentamidine, atovaquone, paromomycin, diclazaril, acyclovir, trifluorouridine, foscarnet, penicillin, gentamicin, ganciclovir, iatroconazole, miconazole, Zn-pyrithione, and silver salts such as chloride, bromide, iodide and periodate.

Growth factor agents that may be incorporated into compositions and devices include, but are not limited to, basic fibroblast growth factor (bFGF), acidic fibroblast growth factor (aFGF), nerve growth factor (NGF), epidermal growth factor (EGF), insulin-like growth factors 1 and 2, (IGF-1 and IGF-2), platelet derived growth factor (PDGF), tumor angiogenesis factor (TAF), vascular endothelial growth factor (VEGF), corticotropin releasing factor (CRF), transforming growth factors α and β (TGF-α and TGF-β), interleukin-8 (IL-8); granulocyte-macrophage colony stimulating factor (GM-CSF); the interleukins, and the interferons.

Other agents that may be incorporated into compositions and devices are acid mucopolysaccharides including, but are not limited to, heparin, heparin sulfate, heparinoids, dermatitin sulfate, pentosan polysulfate, chondroitin sulfate, hyaluronic acid, cellulose, agarose, chitin, dextran, carrageenan, linoleic acid, and allantoin.

Proteins that may be especially useful in the treatment of compromised tissues, such as wounds, include, but are not limited to, collagen, cross-linked collagen, fibronectin, laminin, elastin, and cross-linked elastin or combinations and fragments thereof. Adjuvants, or compositions that boost an immune response, may also be used in conjunction with the wound dressing devices.

Other wound healing agents may include, but are not limited to, metals. Metals such as zinc and silver have long been known to provide excellent treatment for wounds. Delivery of such agents, by the methods and compositions, provide a new dimension of care for wounds.

It is to be understood that in desirable embodiments, the active agents are incorporated into compositions and devices so that the agents are released into the environment. In topical treatments, the agents are then delivered via transdermal or transmucosal pathways. The incorporated agents may be released over a period of time, and the rate of release can be controlled by the amount of cross-linking of the polymers of the matrices. In this way, the matrix retains its ability to affect the local environment, kill or inhibit microorganisms, boost the immune response, exert other alterations of physiological function and provide active agents over an extended period of time.

### EXAMPLES

### Example 1

To illustrate the ability of the closed cell foam matrix to contain and release oxygen, a number of samples were formed. Molds were cast 10.5cm×10.5cm size gel squares. The molds had four wells and were made of polymer or aluminum metal. Each well had a capacity of ˜40g of liquid. The aluminum metal mold was found not to release the gels as easily as the polymer molds.

The superabsorbent polymer material used in each of the samples was obtained from Evonik Stockhausen, LLC (Greensboro, N.C.) under the designation “SR1717” which is manufactured in accordance with U.S. Pat. No. 7,312,286. The superabsorbent material is an aqueous solution of 32% wt/wt oligomeric polyacrylic acid in water where the silanol cross-linker is covalently bonded to the polyacrylic acid chain.

To each 40g sample of the liquid superabsorbent material (SR1717), an amount of 2N sodium hydroxide was added and stirrer as described in Table 1. In samples B-F, 0.14 g of sodium carbonate dissolved in 1 ml of water was added to the superabsorbent polymer mixture and stirred. In Sample A, no sodium carbonate was added. No sign of any bubbles was observed at this stage. An additional amount of hydroxide was added in order to neutralize the acid form of the oligomeric polyacrylic acid in the SR1717.

The mixture was then poured into the mold cell and left overnight in the fume-hood at ambient temperature. The gel was then removed and stored between two layers of sterile wrap. The gel was then cut into four equal pieces and one was taken and placed in the 80° C. oven to dehydrate for 15 minutes. On removal, it was placed on an evaporating dish and an equal weight of 17% hydrogen peroxide, to the weight of the gel, was added to gel. Then, after 1 minute, the sample was turned over to allow the residual peroxide to absorb into the opposite side of the gel. After 3-5 minutes the gel had absorbed all of the peroxide and the sample was then placed into the convection oven for 90 minutes.

A number of samples were formed and test values as illustrated in Table 1.

TABLE 1

| | Wt. of 2N | Initial weight | | | | |
|---|---|---|---|---|---|---|
| | NaOH | of gel | Wt. After | Wt. peroxide | Wt. after 90 | Observations of |
| Sample | added (g) | sample (g) | 80 C. oven | Added (g) | min at 80 C. | foam produced |
| A | 10.5 and no | 3.67 | 2.81 | 2.81 | 2.35 | Quadrupled in size and |
| | Na2CO3 | | | | | doubled in thickness |
| B | 10 | 2.71 | 2.25 | 2.25 | 1.80 | Quadrupled in size and |
| | | | | | | doubled in thickness |
| C | 5 | 2.35 | 1.96 | 1.96 | 1.84 | Doubled in size |
| D | 2.5 | 2.68 | 2.27 | 2.27 | 2.16 | Slight haze of bubbles. |
| | | | | | | Thin foam |
| E | 15 | 2.89 | 2.12 | 2.12 | 1.78 | Made a big puff ball - |
| | | | | | | non uniform cell |
| F | 20 | 2.94 | 2.28 | 2.28 | 2.04 | Made a big puff ball - |
| | | | | | | non uniform cell |
| | | | | | | Gel was still very tacky |
| | | | | | | on removal from the mold |

Sample B was then tested to determine the amount of oxygen released over time. The desired amount of testing material was obtained by cutting the foam using a 19 mm diameter hole puncher. Sample B was then weighed and used for oxygen measurements. All measurements were performed using 15 mL of ultrapure water (diH2O) in a 50 mL conical tube, sealed with parafilm paper. At all times, oxygen measurements were recorded every 10 seconds using the NeoFox® oxygen sensor with the HYOXY probe from Ocean Optics, (Dunedin, Fla.). The baseline was determined by measuring the amount of dissolved oxygen in 15 mL of diH2O at room temperature. Water was then purged with nitrogen gas for 1 minute. Dissolved oxygen was measured after nitrogen purge. Sample B foam was then immersed into the water using tweezers. Release of oxygen by foamed Sample B was measured over time using the NeoFox oxygen sensor. The conical tube was kept sealed at all times to prevent air disturbance.

Sample B was effective at releasing oxygen in water over a total period of 21 hours. Sample B surpassed the baseline level (9.96 ppm) of oxygen within 30 minutes of being in the water. Although initial release of oxygen by Sample B occurred at a fast pace (reaching 30 ppm of oxygen at 3.5 hours), the high levels of oxygen in solution were sustained for a period of up to 21 hours.

This same procedure was performed to obtain oxygen measurements for an OxyGenesys wound dressing obtained from Halyard Health, LLC (Atlanta, Ga.). The capacity of Sample B to release oxygen over time was compared to that of OxyGenesys wound dressing. Unexpectedly, the Sample B foam demonstrated to have a higher oxygen release capacity than the OxyGenesys dressing. At the conditions tested, the sample B foam released a maximum of 645 ppm oxygen per gram of material within an 11 hours' time frame. On the other hand, during this same time frame, the OxyGenesys wound dressing only achieved a maximum of 555 ppm of dissolved oxygen per gram of material (5.4 hours). While at the 11 hours' time point, the Sample B foam was still releasing oxygen, OxyGenesys had already reached its peak and started to decrease.

### Example 2

A aqueous solution was prepared with 40 grams of the superabsorbent material (SR1717), 40 ml water, 10.5 ml 2N sodium hydroxide (a slight excess of base is added in order to neutralize the oligomeric acrylate that is present in the acid form), and 13.6 grams 17% hydrogen peroxide. The sample was poured into a mold 4 mm thick 10.5×10.5cm gel squares. The samples were then cut into four identical squares. Each was infused with an equivalent weight of 17% hydrogen peroxide. Once the material had absorbed all the peroxide liquid the sample was placed in a convection oven at 80oC for 60-90 minutes to generate the foamed sample. Typically the sample doubles in size and thickness during the foam formation.

This sample was then broken up into chunks and placed in a coffee grinder (Smart Grind, model CBGS, Black & Decker, New Britain, Conn.) and processed to obtain white particles which were similar in size to sea salt.

Next, the powder was tested in nitrogen purged water to determine how much oxygen would be delivered by the powder. 0.12g of powder was placed into 50ml of nitrogen sparged water (1.8 ppm oxygen, 19.2oC) and the oxygen released measured (HACH dissolved oxygen (DO) probe, model HQ40d) and found to be 15.2 ppm after 10 minutes and 14.1 ppm after 30 minutes. So it can be seen that converting the foam matrix into a powder does reduce the amount of oxygen delivered, however it is still enough to be a usable product in the powder form.

### Example 3

A aqueous solution was prepared with 40 grams of the superabsorbent material (SR1717), 40 ml water, 10.5 ml 2N sodium hydroxide (a slight excess of base is added in order to neutralize the oligomeric acrylate that is present in the acid form), and 13.6 grams 17% hydrogen peroxide. The sample was poured into a mold 4 mm thick 10.5×10.5cm gel squares. The samples were then cut into four identical squares. Each was infused with an equivalent weight of 17% hydrogen peroxide. Once the material had absorbed all the peroxide liquid the sample was placed in a convection oven at 80oC for 60-90 minutes to generate the foamed sample. Typically the sample doubles in size and thickness during the foam formation.

A 3 mm sample of the foam was cut using a 3 mm punch. The sample weighed 2.9mg. This was placed in 2.5m1 nitrogen sparged PBS solution and gently stirred for 5minutes. The dissolved oxygen level was measured using a NeoFox oxygen sensor with the HYOXY probe (Ocean Optics, Dunedin, Fla.) and measured to be 6.126 ppm. This calculates to 2143 ppm/gram of foam matrix. Desirably, the closed cell foam matrix described herein provides delivers a maximum oxygen release of at least 1500 ppm oxygen per gram of matrix using the test method described above in Example 3.

When introducing elements of the present disclosure or the desirable aspect(s) thereof, the articles “a,” “an,” and “the” are intended to mean that there are one or more of the elements. The terms “comprising,” “including,” and “having” are intended to be inclusive and mean that there can be additional elements other than the listed elements.

The disclosure has been described with reference to various specific and illustrative aspects and techniques. However, it should be understood that many variations and modifications can be made while remaining within the spirit and scope of the disclosure. Many alternatives, modifications and variations will be apparent to those skilled in the art in light of the foregoing description. Accordingly, this disclosure is intended to embrace all such alternatives, modifications, and variations that fall within the spirit and scope of the appended claims.

We claim:

1. A method of forming a closed cell foam matrix containing oxygen comprising:
providing a liquid superabsorbent material, the liquid superabsorbent material comprising:
a. at least 15 percent by mass monoethylenically unsaturated carboxylic, sulphonic or phosphoric acid or salts thereof,
b. an acrylate or methacrylate ester that contains an alkoxysilane functionality,
c. a copolymerizable hydrophilic glycol containing ester monomer;
adding an alkali hydroxide catalyst to form a hydrogel layer;
infusing the hydrogel layer with an oxygen precursor; and
heating and foaming the hydrogel layer to produce oxygen by reacting the alkali hydroxide catalyst and the oxygen precursor, and entrapping the oxygen in the closed cell foam matrix.

2. The method of claim 1 wherein monoethylenically unsaturated carboxylic, sulphonic or phosphoric acid or salts thereof comprises polyacrylic acid.

3. The method of claim 1 wherein the acrylate or methacrylate ester that contains an alkoxysilane functionality comprises methacryloxy-propyl-trimethoxylsilane.

4. The method of claim 1 wherein the copolymerizable hydrophilic glycol containing ester monomer comprises polyethylene glycol.

5. The method of claim 1 wherein the alkali hydroxide catalyst comprises sodium hydroxide.

6. The method of claim 3 wherein the alkali hydroxide catalyst is added at between about 0.5% to about 3% by weight of the liquid superabsorbent material.

7. The method of claim 1 wherein the oxygen precursor comprises hydrogen peroxide.

8. The method of claim 1 wherein the oxygen precursor is added at between about 15% to about 25% by weight of the liquid superabsorbent material.

9. The method of claim 1 wherein a molar ratio of the alkali hydroxide catalyst to the oxygen precursor is in the range of 1.0:0.9 to 0.9:1.0 with the alkali hydroxide catalyst having an additional amount to neutralize the acid component superabsorbent material.

10. The method of claim 1 wherein the superabsorbent material comprises an aqueous solution of an oligomeric polyacrylic acid having a silanol cross-linker covalently bonded to the backbone chain of a polyacrylic acid.

11. The method of claim 1 wherein the closed cell foam matrix delivers oxygen of at least 1500 ppm oxygen per gram of matrix.

12. The method of claim 1 further comprising adding an active agent.

13. A closed cell foam matrix for delivering oxygen, the matrix comprising:
a superabsorbent material comprising:
a. at least 15 percent by mass monoethylenically unsaturated carboxylic, sulphonic or phosphoric acid or salts thereof,
b. an acrylate or methacrylate ester that contains an alkoxysilane functionality,
c. a copolymerizable hydrophilic glycol containing ester monomer; and
oxygen entrapped within the superabsorbent material.

14. The closed cell foam matrix of claim 13 wherein the oxygen is produced by:
adding an alkali hydroxide catalyst to the superabsorbent material to form a hydrogel layer;
infusing the hydrogel layer with an oxygen precursor; and
heating and foaming the hydrogel layer to produce oxygen by reacting the alkali hydroxide catalyst and the oxygen precursor, and entrapping the oxygen in the closed cell foam matrix.

15. The closed cell foam matrix of claim 13 wherein monoethylenically unsaturated carboxylic, sulphonic or phosphoric acid or salts thereof comprises polyacrylic acid.

16. The closed cell foam matrix of claim 13 wherein the acrylate or methacrylate ester that contains an alkoxysilane functionality comprises methacryloxy-propyl-trimethoxylsilane.

17. The closed cell foam matrix of claim 13 wherein the copolymerizable hydrophilic glycol containing ester monomer comprises polyethylene glycol.

18. The closed cell foam matrix of claim 13 wherein can be a sheet, infused/coated in/on a nonwoven, a fiber or a powder form

19. The closed cell foam matrix of claim 13 wherein the alkali hydroxide catalyst comprises sodium hydroxide.

20. The closed cell foam matrix of claim 13 wherein the alkali hydroxide catalyst is added at between about 0.5% to about 3% by weight of the liquid superabsorbent material.

21. The closed cell foam matrix of claim 13 wherein the oxygen precursor comprises hydrogen peroxide.

22. The closed cell foam matrix of claim 13 wherein the oxygen precursor is added at between about 15% to about 25% by weight of the liquid superabsorbent material.

23. The closed cell foam matrix of claim 13 further comprising an active agent.
